(19) United States
(12) Patent Application Publication
(10) Pub. No.: US 2023/0059028 A1
(43) Pub. Date: Feb. 23, 2023

(54) LIFTING COG THREAD

(71) Applicant: FEEL KOREA CO., LTD., Gwangju (KR)

(72) Inventor: Dong Jin KIM, Gwangju (KR)

(73) Assignee: FEEL KOREA CO., LTD., Gwangju (KR)

(21) Appl. No.: 17/488,092

(22) Filed: Sep. 28, 2021

(30) Foreign Application Priority Data
Aug. 18, 2021 (KR) 10-2021-0108642

(51) Int. Cl. A61B 17/06

(52) U.S. Cl. CPC A61B 17/06166; A61B 2017/00792

(57) ABSTRACT

Provided is a lifting cog thread (100) having a predetermined length. The lifting cog thread includes a molded hook portion provided on an outer circumferential surface of the thread. The molded hook portion includes molded recesses (110) and molded protrusions (120) alternating with the molded recesses. The molded hook portion has a spline shape produced by mold processing using a rotational mold processing apparatus. The thread (100) prevents non-uniform pulling in which skin catching force is relatively weakened in a portion of the thread (100).

### TECHNICAL FIELD

The disclosure relates to a lifting cog thread, and more particularly, to a lifting cog thread configured to pierce the skin to reduce wrinkles of the skin or improve sagging of the skin.

### BACKGROUND ART

In general, lifting threads for treatment or surgical suture threads are used in wrinkled portions or sagging portions of the skin. A number of patent applications related to such threads have been filed.

Most patents related to such threads disclose a technology of incising both edge portions of a thread in the longitudinal direction of the thread using a sharp tool so that the incised portions are divided from the remaining portion, thereby forming barb-shaped portions.

However, since most such threads are configured such that the incised portions thereof are divided from both edge portions of the threads, when pulling force is continuously applied to the threads piercing the skin, the incised portions of the barbs may not withstand any further force and may frequently be torn further, inverted, or broken back in the direction of pulling force.

Thus, as the phenomenon of tearing, inversion, or backward breaking occurs, problems, such as the skin being crumpled to have an ugly appearance or severe pain occurring in the skin, may occur. Threads intended to improve the tearing, the inversion, or the backward breaking described above have been disclosed.

Each of these threads is configured such that barbs protrude from both edge portions thereof. Each of the barbs protruding from both edge portions of the thread includes a protrusion having a protruding structure inclined in one direction and a hook integrated with the terminal of the protrusion so as to have a pointed structure and to be bent in the direction of the protrusion.

For this reason, the protrusions serve to apply force resisting the pulling of the skin, and the hooks serve to catch the skin.

Furthermore, since the barbs each including the protrusion and the hook protrude from both edges of the thread, the problem of tearing occurring in barbs formed by a conventional incision method may be overcome. However, since the protrusion and the hook only have a linear structure, the barb may not be able to support itself and properly withstand the pulling force strongly applied to the skin. As a result, the barb may still be inverted or broken back in the direction in which the skin is pulled.

Not only due to the above-described problems, but also since the barbs are formed on both edges of the conventional lifting thread for treatment in the longitudinal direction of the thread, during the lifting performed the thread piercing the skin performs, pulling force may be relatively weak on a portion of the thread without a barb. Thus, the problem that uniform pulling force cannot be obtained has been pointed out.

In addition, a patent disclosed in the patent document of the related art document below relates to a skin lifting thread.

### RELATED ART DOCUMENT

### Patent Documents

Korean Patent No. 10-1655838

### DISCLOSURE

### Technical Problem

The present disclosure has been made keeping in mind the above problems occurring in the related art, and the present disclosure is intended to provide a lifting cog thread used in skin surgery or in lifting treatment for wrinkles and sagging of the skin. The lifting cog thread may pierce the skin, support itself with strong supporting force in the skin without being inverted or broken back by lifting, and ensure uniform pulling force in the skin.

### Technical Solution

In order to realize the above objectives, the present disclosure provides a lifting cog thread (100) having a predetermined length. The lifting cog thread may include a molded hook portion provided on an outer circumferential surface of the thread. The molded hook portion may include molded recesses (110) and molded protrusions (120) alternating with the molded recesses. The molded hook portion may have a spline shape produced by mold processing using a rotational mold processing apparatus.

The molded recesses (110) and the molded protrusions (120) alternating with the molded recesses may be mold-processed to be sequentially connected.

Each of the molded protrusions (120) alternating with the molded recesses (110) may have a complex hook structure on either end thereof. The complex hook structure may include a first hook (122) and a second hook (123) provided sequentially.

The molded recesses (110) and the molded protrusions (120) alternating with the molded recesses may be mold-processed while being rotated around the outer circumferential surface of the thread (100) at an angle of rotation of from 1° to 180°.

The mold processing machine may be configured to turn around a mold and perform mold processing on the molded hook portion by punching the outer circumferential surface of the thread (100) while rotating at a predetermined angle of rotation. The mold may include a mold inlet extending through a central portion of the mold to allow the thread (100) to be inserted therethrough in a horizontal position and a helix extending on an outer circumferential surface of the mold inlet.

Also provided is a lifting cog thread (100) having a predetermined length. The lifting cog thread may include a molded hook portion provided on an outer circumferential surface of the thread. The molded hook portion may include molded recesses (110) and molded protrusions (130) alternating with the molded recesses. Each of the molded protrusions may include a mediation adjustment hook (131) in a central portion thereof. The molded hook portion may have a spline shape produced by mold processing using a rotational mold processing apparatus.

The molded recesses (110) and the molded protrusions (130) alternating with the molded recesses may be mold-processed to be sequentially connected.

Each of the molded protrusions (130) alternating with the molded recesses (110) may have a complex hook structure on either end thereof. The complex hook structure may include a first hook (132) and a second hook (133) provided sequentially.

The molded recesses (110) and the molded protrusions (130) alternating with the molded recesses may be mold-processed while being rotated around the outer circumferential surface of the thread 100 at an angle of rotation of from 1° to 180°.

### Advantageous Effects

According to the present disclosure, the lifting cog thread can prevent the barb portions from being torn, inverted, or broken back in the direction in which the skin is pulled, occurring in a barb thread fabricated by incising both edge portions of a conventional thread or a conventional lifting thread fabricated by forming protrusions on both edge portions of a thread, thereby sustaining itself with strong supporting force regardless of strong skin-pulling force caused by piercing into the skin.

In addition, according to the present disclosure, since the lifting cog thread can properly support itself regardless of strong skin-pulling force caused by piercing into the skin, it is possible to maintain the improved shape of the skin in which the wrinkles are reduced or the sagging of the skin is improved by skin-lifting treatment.

In addition, according to the present disclosure, since the lifting cog thread can properly support itself regardless of strong skin-pulling force caused by piercing into the skin, it is possible to prevent the barb portions from being torn, inverted, or broken back in the direction in which the skin is pulled, it is possible to prevent the skin from being crumpled into an ugly appearance and reduce pain in the skin.

Furthermore, according to the present disclosure, since the molded recesses and the molded protrusions of the molded hook portion are uniformly over the outer circumferential surface of the thread, in the lifting operation of the thread piercing the skin, it is possible to apply uniform pulling force to the skin so as to uniformly smooth the entire wrinkled portion or the sagged portion of the skin, thereby producing fresh skin that appears natural rather than artificial

### BRIEF DESCRIPTION OF DRAWINGS

FIG. 1 is a schematic perspective view illustrating a lifting cog thread according to a first embodiment of the present disclosure;

FIG. 2 is a schematic view illustrating the cross-sectional shape of the molded hook portions of the lifting cog thread according to the first embodiment of the present disclosure;

FIG. 3 is a schematic view illustrating the cross-sectional shape of the molded hook portions of the cog thread illustrated in FIG. 2, the molded hook portions being symmetrical about a point line;

FIG. 4 is a schematic view illustrating a lifting cog thread according to a second embodiment of the present disclosure, with the cross-sectional shape of molded hook portions of the cog thread being symmetrical about a point line;

FIG. 5 a schematic view illustrating the cross-sectional shape of molded hook portions of a lifting cog thread according to a third embodiment of the present disclosure;

FIG. 6 is a schematic view illustrating the cross-sectional shape of the molded hook portions of the cog thread illustrated in FIG. 5, the molded hook portions being symmetrical about a point line;

FIG. 7 is a schematic view illustrating the cross-sectional shape of an example of the thread in which the molded hook portions are formed on the outer circumferential portions of the thread at a predetermined angle of rotation using the mold processing machine; and

FIG. 8 is a schematic view illustrating the cross-sectional shape of another example of the thread in which the molded hook portions are formed on the outer circumferential portions of the thread at a predetermined angle of rotation using the mold processing machine.

### MODE FOR INVENTION

The present disclosure will now be described in detail with reference to the accompanying drawings. It will be apparent to those skilled in the art that a variety of modifications and variations are possible without departing from spirit and scope of the present disclosure. Rather, these embodiments are provided so that the present disclosure will be thorough and complete and will fully convey the scope of the present disclosure to those skilled in the art, and the scope of the present disclosure shall be defined by the appended claims and equivalents thereof.

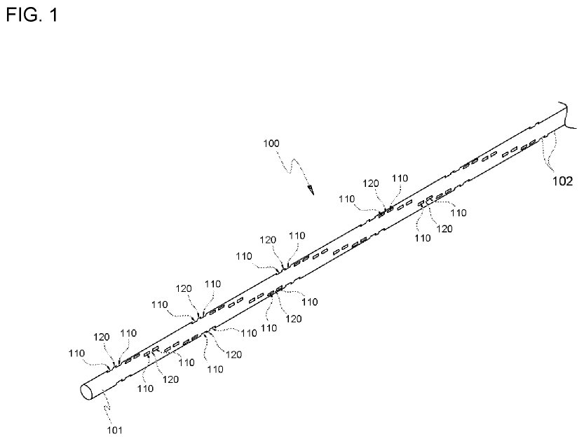

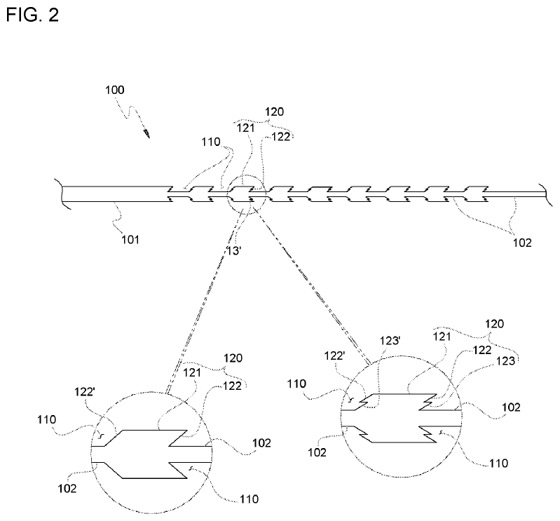

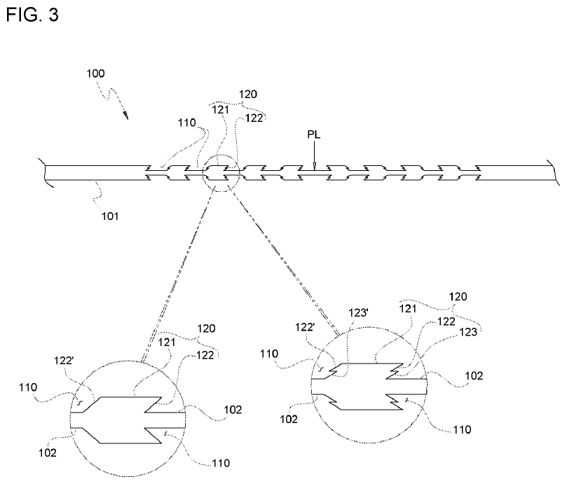

In a lifting cog thread 100 according to a first embodiment of the present disclosure, as illustrated in FIGS. 2 and 3 with reference to, for example, FIG. 1, a molded hook portion including molded recesses 110 and molded protrusions 120 alternating with the molded recesses 110 may be formed on the outer circumferential surface of the thread 100 having a predetermined length. The molded hook portion may be formed in the shape of a spline or the like by mold processing using a rotational mold processing apparatus.

The molded recesses 110 and the molded protrusions 120 alternating with the molded recesses 110 may be mold-processed so as to be sequentially connected. Thus, the molded recesses 110 and the molded protrusions 120 are configured to be uniformly distributed over the entire surface of the thread 100.

That is, the molded recesses 110 and the molded protrusions 120, alternating with the molded recesses, may be mold-processed using the mold processing apparatus while being rotated around the outer circumferential surface of the thread 100, thereby being uniformly distributed over the entire surface of the thread 100.

Both ends of each of the molded protrusions 120 alternating with the molded recesses 110 have a complex hook structure comprised of a first hook 122 and a second hook 123 formed sequentially. The first hook 122 and the second hook 123 may also form an inclined cascading structure.

Due to the cascading structure, the first hook 122 and the second hook 123 described above may have a complementary function to prevent a phenomenon, such as inversion, due to the skin being pulled strongly. In addition, one of the first hook 122 and the second hook 123 may continuously maintain the catching function.

On the opposite sides of the first hooks 122 and the second hooks 123, third hooks 122′ and fourth hooks 123′ corresponding to the first hooks 122 and the second hooks 123 may be formed. In some cases, only the third hooks 122′ having a single slope may be provided. The third and fourth hooks 122′ and 123′ or the third hooks 122′ described above may prevent slipping that may occur in the opposite direction to the lifting.

In particular, the molded protrusions 120 alternating with the molded recesses 110 may be mold-processed while being rotated around the outer circumferential portions of the thread 100 at an angle of rotation of from 1° to 180° using a rotational mold processing machine rotating around the outer circumferential portion of a mold.

The molded recesses 110 and the molded protrusions 120 alternating with the molded recesses 110 may be mold-processed while being rotated around the outer circumferential portions of the thread 100 at the angle of rotation of from 1° to 180°, on the basis of the angle of rotation set in the mold processing apparatus.

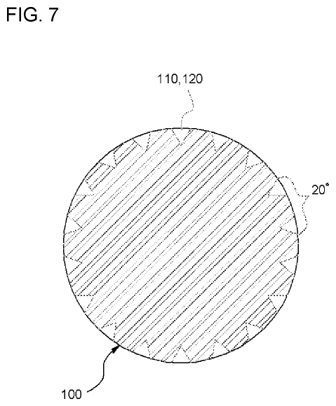

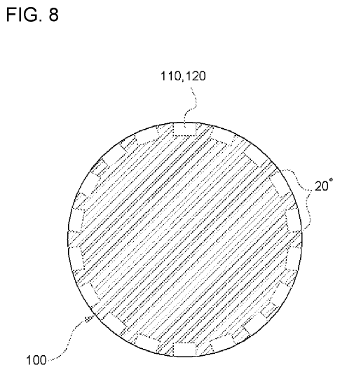

As illustrated in FIG. 7 or 8, the molded hook portion including the molded recesses 110 and the molded protrusions 120 alternating with the molded recesses 110 may be formed on the basis of the angle of rotation (e.g., 20°) set in the mold processing apparatus while being rotated around the outer circumferential surface of the thread 100. The angle of rotation may be freely adjusted in the range of 1° to 180°.

In particular, the mold processing apparatus may be implemented as a mold processing machine configured to punch the outer circumferential portions of the thread 100 while rotating around the thread 100 at a predetermined angle of rotation within a chamber having a predetermined size.

Although the mold processing machine provided within the chamber of the mold processing apparatus is not illustrated in the drawings, it should be understood that the structure of the mold processing machine is clearly apparent from the following description thereof.

The mold processing machine may include a mold, a rotating-movement driven body, a rotating-movement driving body, a rotating driving means, and a straight driving means. The mold may include a mold inlet extending through the central portion of the mold in a horizontal longitudinal direction thereof such that the thread 100 may be introduced into the mold inlet in a horizontal and tight position and a helix extending around the outer circumferential surface of the mold inlet in the longitudinal direction thereof. The mold may have a cylindrical structure having a predetermined length.

That is, outer circumferential portions of the thread 100 inserted into the mold inlet of the mold are punched by punching blades driven by power supplied from the straight driving means so as to form the molded hook portions (i.e., the components including the molded recesses 110 and the molded protrusions 120 alternating with the molded recesses 110).

The thread 100 capable of maintaining tight tension while being inserted into the mold inlet may be realized by an unwinding roller, a winding roller, and a tension sensor provided on one side and the other side of the chamber.

The rotating-movement driven body is configured to be inserted into a peripheral portion of the mold and is connected to the helix by engagement. The rotating-movement driven body may be moved while being rotated at a predetermined angle in the longitudinal direction of the mold. The rotating-movement driven body may be implemented as, for example, a spur gear but is not limited thereto.

In particular, the punching blades including the straight driving means may be provided on both edge portions or one or the other edge portion of the rotating-movement driven body. The straight driving means may be implemented as, for example, a cylinder suitable to provide straight drive power but is not limited thereto.

A pair of the cylinders may be coupled to both edges of the rotating-movement driven body in a symmetrical structure such that the punching blades are coupled to the distal end of the piston.

When the rotating-movement driven body is stopped after having been rotated at the predetermined angle of rotation, the cylinders may be operated. Advancement/retreat paths may be provided in some portions of the mold to extend therethrough. The punching blades moving through the forward movement of the piston in response to the operation of the cylinder may reach the thread 100 located in the central portion of the mold through the advancement/retreat paths.

The rotating-movement driving body is connected to the outer circumferential surface of the rotating-movement driven body by engagement. The rotating-movement driving body may be rotated and moved by a distance by which the rotating-movement driven body moves while serving to transfer rotating power by which the rotating-movement driven body can rotate along the helix of the mold.

Although the rotating driving means may be implemented as, for example, a motor, the rotating driving means is not limited thereto and may include any power means necessary for rotation. The rotating driving means may couple the central portion of the rotating-movement driving body to the rotor shaft of the motor, may be a structure disposed in a position adjacent to the mold and capable of horizontal sliding movement, and provide rotating power necessary for the rotation of the rotating-movement driving body.

In addition, although the molded recesses 110 and the molded protrusions 120 alternating with the molded recesses 110 may be formed on the outer circumferential portions of the thread by a mold processing method, the molded recesses 110 and the molded protrusions 120 may be formed by a non-molding method. The mold processing method may be implemented as, for example, a punching method using the punching blades, and the non-molding method may be implemented as a laser processing method of emitting a laser beam as set in a program.

Referring to the figures, the molded recesses 110 and the molded protrusions 120 alternating with the molded recesses 110 are formed, for example, on the outer circumferential surface of the thread 100 by mold processing, and may be formed on the left side and the right side of a point line PL serving as a reference point to have a symmetrical structure. In the skin, the molded recesses 110 may provide spaces to be filled with skin.

The molded protrusions 120 may withstand lifting occurring, for example, in skin, i.e., lifting pulling force acting in the rightward direction, with strong resistance so as not to be inverted or broken back. The molded protrusions 120 may also withstand lifting occurring, for example, in the skin, i.e., lifting pulling force acting in the leftward direction, with strong resistance so as not to be inverted or broken back.

In the above-described lifting cog thread according to the first embodiment, since the molded recesses 110 and the molded protrusions 120 are formed while being rotated around the outer circumferential surface of the thread 100 at the predetermined angle of rotation, as shown, in particular, from FIG. 1, the molded recesses 110 and the molded protrusions 120 may be uniformly distributed around the outer circumferential surface of the thread 100. Thus, the uniformity of catching is obtained during the lifting-pulling action, the entirety of the wrinkles of the skin or the entirety of the sagging portion of the skin is uniformly smoothed. Accordingly, it is possible to produce fresh skin that appears natural rather than artificial.

In addition, the molded hook portion including the molded recesses 110 and the molded protrusions 120 alternating with the molded recesses 110 may be formed using a mold processing machine having a different structure by rotating the thread 100.

The mold processing machine may include: rubber holding pieces pulling both end portions of the thread 100 having a predetermined length so that the thread 100 is tensioned; motors rotating the rubber holding pieces; the mold in which the punching is performed when the thread 100 is inserted thereto; and the punching blade configured to punch the outer circumferential portions of the thread 100.

It should be understood, however, that the structure systematically connecting the rubber holding pieces, the motors, the mold, the straight driving means, and the punching blade is clearly apparent from the foregoing description and thus will not be illustrated in the drawings.

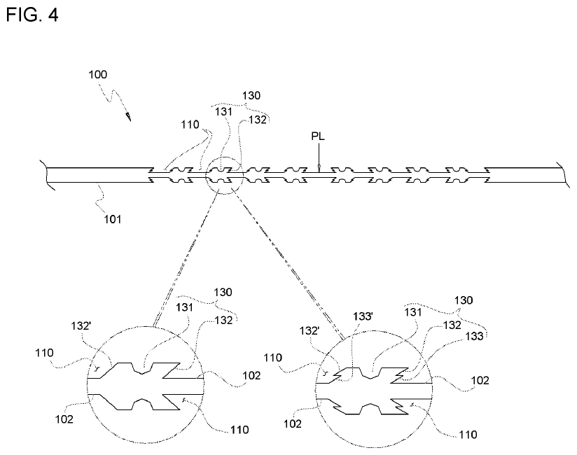

In addition, in a lifting cog thread 100 according to a second embodiment of the present disclosure illustrated, for example, in FIG. 4, the molded hook portion including the molded recesses 110 and molded protrusions 130 alternating with the molded recesses 110, with a mediation adjustment hook 131 being formed on the central portion of each of the molded protrusions 130, may be formed on the outer circumferential surface of the thread 100 having a predetermined length. The molded hook portion may be formed in the shape of a spline or the like by mold processing using a rotational mold processing apparatus.

In particular, the mediation adjustment hook 131 may perform a mediation function to adjust catching so that the thread is not easily pulled by pulling force acting in the opposite direction to the catching caused by lifting pulling. That is, even in the case that the catching is performed by the lifting pulling acting in one direction of the molded protrusions 130, the catching may be adjusted so as not to be easily pulled when pulled in the opposite direction depending on a variety of lipid structures of the inner layer of the skin.

Each of the molded protrusions 130 alternating with the molded recesses 110 has a complex hook structure in which a first hook 132 and a second hook 133 are sequentially formed so as to form an inclined cascading structure.

Due to the cascading structure, the first hook 132 and the second hook 133 described above may have a complementary function to prevent a phenomenon, such as inversion, due to the skin being pulled strongly. In addition, one of the first hook 132 and the second hook 133 may continuously maintain the catching function.

On the opposite sides of the first hooks 132 and the second hooks 133, third hooks 132′ and fourth hooks 133′ corresponding to the first hooks 132 and the second hooks 133 may be formed. In some cases, the third hooks 132′ having a single slope may be only provided. The third and fourth hooks 132′ and 133′ or the third hooks 132′ described above may prevent slipping that may occur in the opposite direction to the lifting.

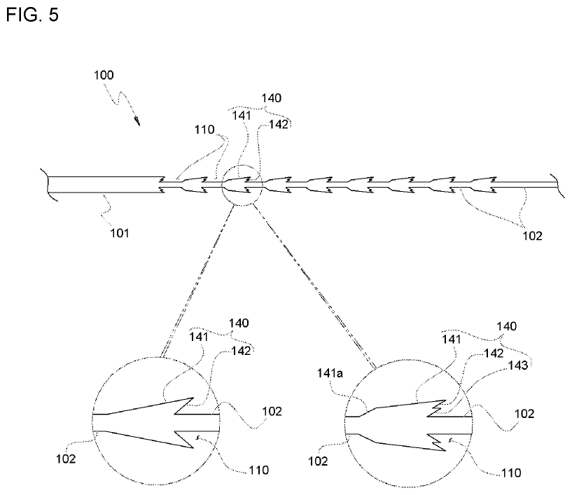

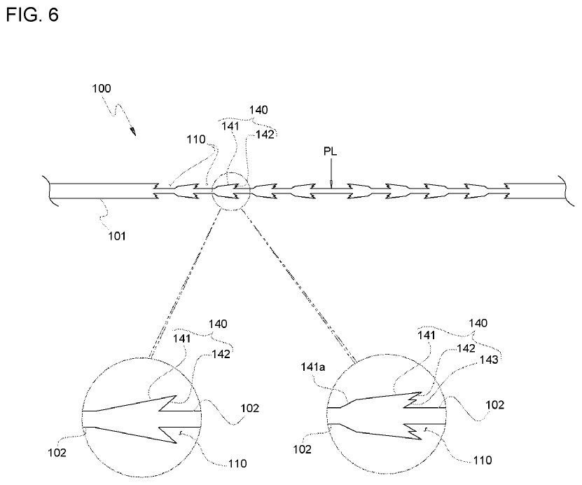

In a lifting cog thread 100 according to a third embodiment of the present disclosure, as illustrated in FIGS. 5 and 6, a molded hook portion including the molded recesses 110 and molded protrusions 140 alternating with the molded recesses 110 may be provided on the outer circumferential surface of the thread 100 having a predetermined length. The molded hook portion may be formed in the shape of a spline or the like by mold processing using a rotational mold processing apparatus.

Each of the molded protrusions 140 may protrude such that two lines 141 and 141a thereof are connected at a bend on one side. The two lines 141 and 141a may serve to promote smooth piercing in the piercing direction. Only a single line 141 of the two lines 141 and 141a may be provided. The two lines 141 and 141a or the single line 141 may prevent slipping that may occur in the opposite direction to the lifting.

In addition, each of the molded protrusions 140 may have a complex cascading structure comprised of hooks 142 and 143 enabling a hooking action in the direction of the lifting.

The thread 100 described above may be made from a biodegradable substance, e.g., p-dioxanone (PDO), ε-caprolactone (PCL), L-lactide (PLLA), glycolide (PGA), L-lactide and glycolide (PLGA), glycolide and s-caprolactone (PGCL), DL-lactide (PDLLA), DL-lactide, glycolide (PDLGA), or a combination of at least two thereof.

At least two among the biodegradable substances, such as p-dioxanone (PDO), ε-caprolactone (PCL), L-lactide (PLLA), glycolide (PGA), L-lactide and glycolide (PLGA), glycolide and ε-caprolactone (PGCL), DL-lactide (PDLLA), DL-lactide, and glycolide (PDLGA), may be mixed at different weight ratios depending on the intended purpose of use.

The above-described thread 100 may be mold processed on the circular outer circumferential surface. In some cases, the thread 100 may be mold processed along the outer circumference surface deformed to be flat, for example, the rectangular outer circumference surface.

1. A lifting cog thread (100) having a predetermined length, the lifting cog thread comprising a molded hook portion provided on an outer circumferential surface of the thread, wherein the molded hook portion comprises molded recesses (110) and molded protrusions (120) alternating with the molded recesses, and has a spline shape produced by mold processing using a rotational mold processing apparatus.

2. The lifting cog thread of claim 1, wherein the molded recesses (110) and the molded protrusions (120) alternating with the molded recesses are mold-processed to be sequentially connected.

3. The lifting cog thread of claim 2, wherein each of the molded protrusions (120) alternating with the molded recesses (110) has a complex hook structure on either end thereof, the complex hook structure comprising a first hook (122) and a second hook (123) provided sequentially.

4. The lifting cog thread of claim 1, wherein the molded recesses (110) and the molded protrusions (120) alternating with the molded recesses are mold-processed while being rotated around the outer circumferential surface of the thread (100) at an angle of rotation of from 1° to 180°.

5. The lifting cog thread of claim 4, wherein the mold processing machine is configured to turn around a mold and perform mold processing on the molded hook portion by punching the outer circumferential surface of the thread (100) while rotating at a predetermined angle of rotation, wherein the mold comprises a mold inlet extending through a central portion of the mold to allow the thread (100) to be inserted therethrough in a horizontal position and a helix extending on an outer circumferential surface of the mold inlet.

6. A lifting cog thread (100) having a predetermined length, the lifting cog thread comprising a molded hook portion provided on an outer circumferential surface of the thread, wherein the molded hook portion comprises molded recesses (110) and molded protrusions (130) alternating with the molded recesses, each of the molded protrusions comprising a mediation adjustment hook (131) in a central portion thereof, and the molded hook portion has a spline shape produced by mold processing using a rotational mold processing apparatus.

7. The lifting cog thread of claim 6, wherein the molded recesses (110) and the molded protrusions (130) alternating with the molded recesses are mold-processed to be sequentially connected.

8. The lifting cog thread of claim 7, wherein each of the molded protrusions (130) alternating with the molded recesses (110) has a complex hook structure on either end thereof, the complex hook structure comprising a first hook (132) and a second hook (133) provided sequentially.

9. The lifting cog thread of claim 6, wherein the molded recesses (110) and the molded protrusions (130) alternating with the molded recesses are mold-processed while being rotated around the outer circumferential surface of the thread 100 at an angle of rotation of from 1° to 180°.
